(19) United States
(12) Patent Application Publication
(10) Pub. No.: US 2008/0171884 A1
(43) Pub. Date: Jul. 17, 2008

(54) Novel Polymorph Form G of Fluvastatin Sodium and Process For the Preparation Thereof

(76) Inventors: Venkatasubramanian Radhakrishnan Tarur, Mulund (IN); Dhananjay Govind Sathe, Maharashtra (IN); Narayana Rao Mantripragada, Maharashtra (IN); Rajesh Bhopalkar, Maharashtra (IN); Kamlesh Digambar Sawant, Maharashtra (IN); Dattatraya Nivrutti Chavan, Maharashtra (IN)

Correspondence Address: KLEIN, O'NEILL & SINGH, LLP, 43 CORPORATE PARK, SUITE 204, IRVINE, CA, 92606, US

(21) Appl. No.: 11/908,846

(22) PCT Filed: Jun. 3, 2005
(86) PCT No.: PCT/IN2005/000181

§ 371 Date: Sep. 17, 2007

(30) Foreign Application Priority Data
Mar. 22, 2005 (IN) 317/MUM/2005

(51) Int. Cl. C07D 209/04

(52) U.S. Cl. 548/491

(57) ABSTRACT

A Polymorph Form G of Fluvastatin sodium having X-ray diffraction peak at 2θ value of 3.48 is provided. A process for the preparation of the novel polymorph Form G comprising spray drying a 3 to 10% w/v solution of Fluvastatin sodium in a solvent using a spray gun at inlet temperature of 120 to 200° C. and outlet temperature of 60 to 110° C. of the spray gun is also provided.

### FIELD OF INVENTION

This invention relates to novel polymorph Form G of Fluvastatin sodium and process for the preparation thereof.

Fluvastatin sodium is a common name for (±)-7-[3-(4-fluorophenyl)-1-(1-methylethyl)-1H-indol-2-yl]-3,5-dihydroxy-6-heptenoic acid mono sodium salt of the following formula 1:

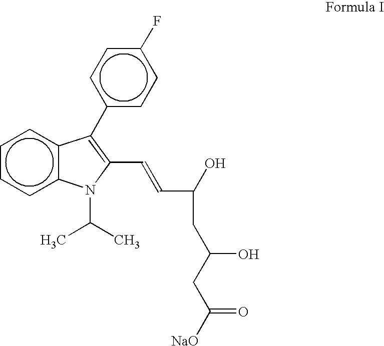

Fluvastatin sodium is a racemic mixture of 3R,5S and 3S,5R-dihydroxy enantiomer of the Formula 1 and is used as anti-hypercholesterolemic, anti-hyperlipoproteinemic and anti-atherosclerotic agent. Fluvastatin is an inhibitor of 3-hydroxy-3-methyl-glutaryl coenzyme A (HMG-CoA) reductase, which is an enzyme used in the regulation of cholesterol biosynthesis.

### BACKGROUND OF INVENTION

Fluvastatin and its sodium salt are disclosed in U.S. Pat. No. 4,739,073 (Column 50). Fluvastatin sodium is isolated by lyophilization. U.S. Pat. Nos. 5,189,164 and 5,354,772 and European Patent No 0114027 also describe preparation of Fluvastatin sodium by lyophilization step B of Example 1 in column 19 of U.S. Pat. No. 5,189,164 and step B of Example 6 in column 58 of U.S. Pat. No. 5,354,772 and Example 5 of European Patent No 0114027). PCT Publication No WO-A-97/49681 and its equivalent U.S. Pat. No. 6,124,340 teach that lyophilization of Fluvastatin sodium yields a mixture of crystalline Form A and amorphous material and discloses a new crystalline Form B of Fluvastatin sodium. Form B is obtained by transformation, of material containing Form A in a slurry of a mixture of an organic solvent and water or by crystallization from an organic solvent and water mixture. U.S. Pat. No. 6,696,479 describes preparation of highly crystalline Form A of Fluvastatin sodium by lyophilization. It also describes the conversion of Form A into different hydrates thereof named C, D, E and F by exposing Form A to different humidity conditions. PCT publication No WO2004/096765 discloses novel Form BA of Fluvastatin sodium using aliphatic ethers as antisolvents. It also describes the use of corresponding free acid or the ester or a salt of Fluvastatin sodium as the starting material. PCT Publications Nos WO2004/113291 and WO2004/113292 relate to novel polymorph Forms I to CV of Fluvastatin prepared by solvent crystallization.

### OBJECT OF INVENTION

An objection of the invention is to provide a novel polymorph Form G of Fluvastatin sodium which is very pure and stable.

Another object of the invention is to provide a process for the preparation of a novel polymorph Form G of Fluvastatin sodium which is simple, easy and convenient to carry out.

Another object of the invention is to provide a process for the preparation of a novel polymorph Form G of Fluvastatin sodium which consumes reduced, amount of energy and is economical.

Another object of the invention is to provide a process for the preparation of a novel polymorph Form G of Fluvastatin sodium which can be carried out continuously and is industrially viable.

### DETAILED DESCRIPTION OF INVENTION

According to the invention there is provided, novel polymorph Form G of Fluvastatin sodium having X-ray diffraction peak at 2θ value of 3.48.

According to the invention there is also provided a process for the preparation of a novel polymorph Form G of Fluvastatin sodium having X-ray diffraction peak at 2θ value of 3.48 comprising spray drying a 3 to 10% w/v solution of Fluvastatin sodium in a solvent using a spray gun at inlet temperature of 120 to 200° C. and outlet temperature of 60 to 110° C. of the spray gun.

Any liquid substance which has capacity to dissolve Fluvastatin sodium at room temperature or higher temperatures can be used as solvent to form solution of Fluvastatin sodium. The solvent is selected, for example, from water or lower alcohol such as methanol or ethanol, preferably water.

Preferably a 9% solution of Fluvastatin sodium is spray dried. Preferably spray drying of the solution of Fluvastatin sodium is carried out at inlet temperature of 145 to 185° C. and outlet temperature of 60 to 100° C. of the spray gun.

According to the invention there is provided novel polymorph Form G of Fluvastatin sodium which is very stable and pure and is suitable for dosage development as purity and stability are favourable dosage requirements. The process of the invention comprises spray drying of a solution of Fluvastatin sodium using a spray gun which is easy to handle, requires negligible repairs and maintenance and consumes reduced energy. Therefore, the process of the invention is very simple, easy and convenient to carryout. It requires reduced amount of energy and is economical. Besides, it can be carried out continuously and is industrially viable.

The following experimental examples are illustrative of the invention but not limitative of the scope thereof.

### EXAMPLE 1

An aqueous solution of Fluvastatin sodium at a concentration of 9% weight/volume was spray dried by a spray gun (PSD 00 Pilot, Hemraj, India at pressure 500 to 600 psi and flow rate of 2 Lts/hr) at an inlet temperature of 185° C. and outlet temperature of 100° C. of the spray gun. Purity of the product obtained was 99.88%.

### EXAMPLE 2

The procedure of Example 1 was carried out at inlet temperature of 165° C. and outlet temperature of 80° C. of the spray gun. Purity of the product obtained was 99.58%.

### EXAMPLE 3

The procedure of Example 1 was carried out at inlet temperature of 145° C. and outlet temperature of 60° C. of the spray gun. Purity of the product obtained was 99.32%.

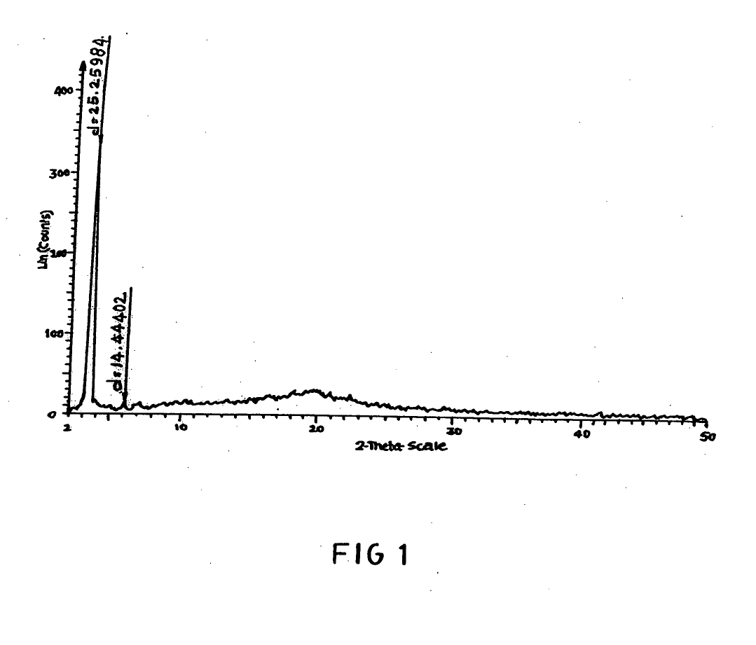

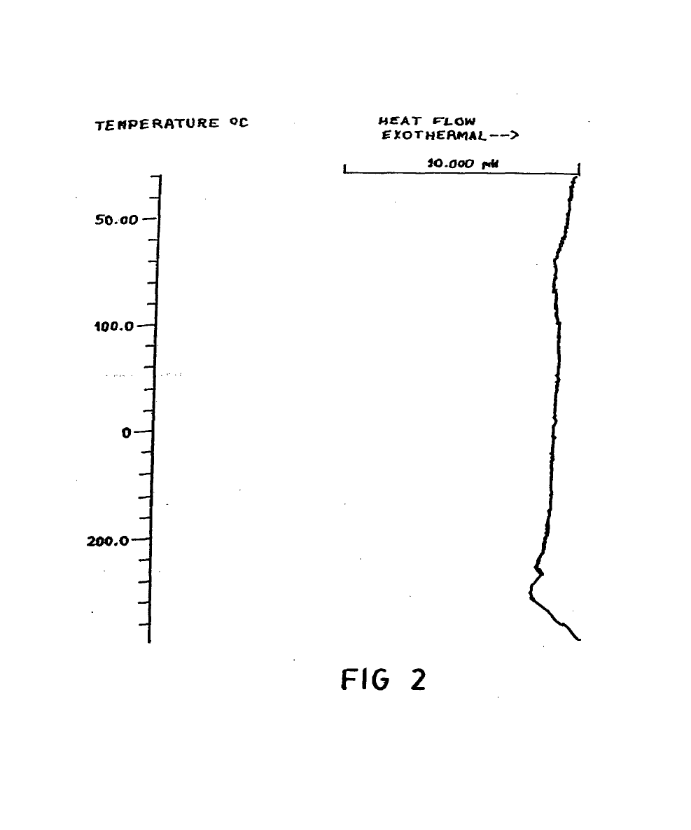

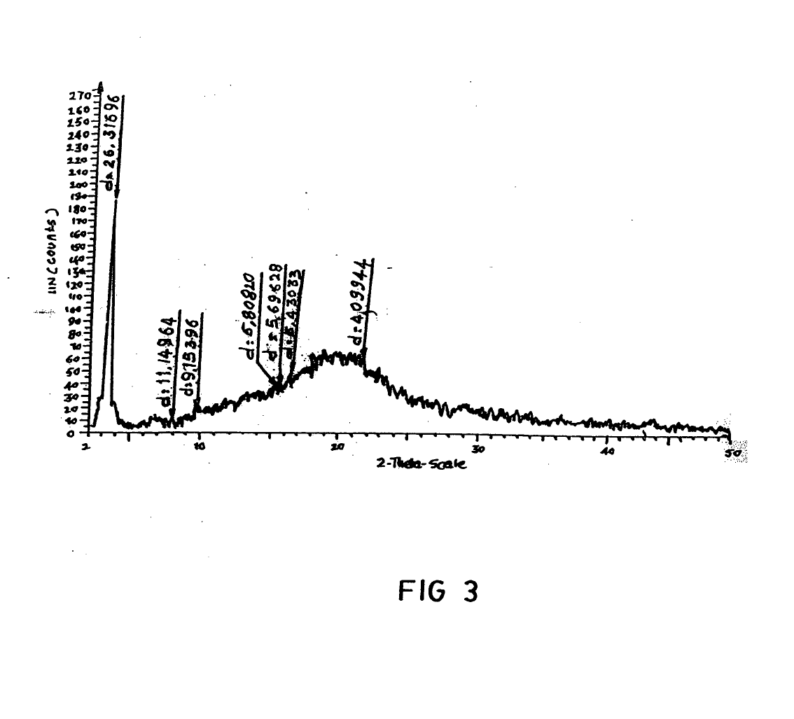

X-Ray Powder Diffraction (XRPD) pattern of the product of Examples 1 to 3 was obtained on D 8-Advance, Bruker AXE, Germany, diffractometer equipped with Scintillation detector using Copper Kα (λ=1.5406 Å) radiation with scanning range between 2-50 θ at scanning speed of 2°/min. The XRPD pattern showed single sharp X-ray diffraction peak at 2θ value of 3.48 as shown in FIG. 1 of the accompanying drawings which is characteristic of the product. Differential Scanning Calorimeter (DSC) of the product was performed on Mettler DSC 20 instrument. Samples of 2 mg to 3 mg weighed in aluminum crucibles with holes were scanned at a heating rate of 11° C. per minute under nitrogen atmosphere at rate of 35 ml/min. The product exhibited small endotherm at 226.3° C. in DSC as shown in FIG. 2 of the accompanying drawings which is also characteristic of the product. Because of the above characteristics the product is considered to be novel and is named as polymorph Form G of Fluvastatin sodium. Form G was found to be stable when exposed to 45% relative humidity at 20° C. for one week as shown by the XPRD pattern taken after one week as shown in FIG. 3 of the accompanying drawings.

1. A Polymorph Form G of Fluvastatin sodium having X-ray diffraction peak at 2θ value of 3.48.

2. A process for the preparation of a polymorph Form G of Fluvastatin sodium having X-ray diffraction peak at 2θ value of 3.48, comprising:
spray drying a 3 to 10% w/v solution of Fluvastatin sodium in a solvent using a spray gun, wherein inlet temperature of the spray gun is between 120 to 200° C., and outlet temperature of the sprav gun is between 60 to 110° C.

3. A process as claimed in claim 2, wherein the solvent is selected from the group consisting of water or a lower alcohol such as methanol or ethanol.

4. A process as claimed in claim 2, wherein the solvent is water.

5. A process as claimed in claim 2, wherein a 9% w/v solution of Fluvastatin sodium is spray dried.

6. A process as claimed in claim 2, wherein the inlet temperature of the spray gun is between 145 to 185° C. and the outlet temperature is between 60 to 100° C.
